(19) United States
(12) Patent Application Publication
(10) Pub. No.: US 2026/0115474 A1
(43) Pub. Date: Apr. 30, 2026

(54) SYSTEMS AND METHODS FOR EXTERNAL CONTROL OF POWER TRANSMISSION TO, AND STIMULATION THERAPY BY, AN IMPLANTED NEUROMODULATION DEVICE

(71) Applicant: NERVONIK INC., Los Angeles, CA (US)

(72) Inventors: Barry Keenan, Las Vegas, NV (US); Aydin Babakhani, Los Angeles, CA (US)

(21) Appl. No.: 19/144,039

(22) PCT Filed: Jan. 22, 2024
(86) PCT No.: PCT/US2024/012347

§ 371 Date: Jun. 27, 2025

Related U.S. Application Data
(60) Provisional application No. 63/440,818, filed on Jan. 24, 2023.

(51) Int. Cl. A61N 1/36; A61N 1/372

(52) U.S. Cl. CPC A61N 1/36139; A61N 1/36125; A61N 1/3614; A61N 1/36178; A61N 1/37235

(57) ABSTRACT

A neuromodulation system includes a neuromodulation device configured to be implanted at an implant site and an external device configured to be positioned at an external sensing site relative to the neuromodulation device. The neuromodulation device and external device are configured to implement a neuromodulation therapy method, wherein the external device transmits an RF signal to the neuromodulation device; the neuromodulation device delivers a neuromodulation therapy comprising a stimulation pulse at the implant site based on the RF signal; and the external device obtains a measurement that results from delivery of the stimulation pulse, and initiates a change in the neuromodulation therapy based on the measurement.

### TECHNICAL FIELD

The present disclosure relates generally to neuromodulation systems and methods, and more particularly, to systems and methods for external control of power transmission to and implanted neuromodulation device, and control of stimulation therapy delivery by an implanted neuromodulation device.

### BACKGROUND

Peripheral nerve stimulation (PNS) may be used to treat a range of chronic disorders, such as neuropathic pain and urinary disfunction. A typical neuromodulation system for PNS includes an implantable component having electrodes that are placed next to a target nerve and an external component. Energy is transmitted from the external component to the implanted component, which uses the energy to deliver neuromodulation therapy, e.g., electrical stimulation pulses.

### SUMMARY

In one aspect of the disclosure, a neuromodulation system includes a neuromodulation device configured to be implanted at an implant site and an external device configured to be positioned at an external sensing site relative to the neuromodulation device. The neuromodulation device and external device are configured to implement a neuromodulation therapy method, wherein the external device transmits an RF signal to the neuromodulation device; the neuromodulation device delivers a neuromodulation therapy comprising a stimulation pulse at the implant site based on the RF signal; and the external device obtains a measurement that results from delivery of the stimulation pulse, and initiates a change in the neuromodulation therapy based on the measurement.

In another aspect of the disclosure, a neuromodulation system includes a neuromodulation device configured to be implanted at an implant site and an external device configured to be positioned at an external site relative to the neuromodulation device. The neuromodulation device and external device are configured to implement a neuromodulation therapy method, wherein the external device transmits an RF signal to the neuromodulation device; the neuromodulation device delivers a neuromodulation therapy comprising a stimulation pulse at the implant site based on the RF signal; and the external device senses electrical activity of tissue resulting from delivery of the stimulation pulse. The sensed electrical activity includes a stimulation response comprising an evoked stimulation response, a stimulation artifact, and an ECAP response. The external unit determines a measurement based on the ECAP response and the evoked stimulation response, and initiates a change in the neuromodulation therapy based on the measurement.

In another aspect of the disclosure, a neuromodulation system includes a neuromodulation device configured to be implanted at an implant site and an external device configured to be positioned at an external site relative to the neuromodulation device. The neuromodulation device and external device are configured to implement a neuromodulation therapy method, wherein the external device transmits an RF signal to the neuromodulation device; the neuromodulation device delivers a neuromodulation therapy comprising a stimulation pulse at the implant site based on the RF signal; and the external device obtains a signal waveform representing neural activity that results from delivery of the stimulation pulse, and initiates a change in the neuromodulation therapy based on the signal waveform.

In yet another aspect of the disclosure, a neuromodulation system includes a neuromodulation device configured to be implanted at an implant site; and an external device configured to be positioned at an external site relative to the neuromodulation device. The neuromodulation device and external device are configured to implement a neuromodulation therapy method, wherein the external device transmits an RF signal to the neuromodulation device; the neuromodulation device delivers a neuromodulation therapy comprising a stimulation pulse at the implant site based on the RF signal; and the external device obtains a measurement of skin conductivity that results from delivery of the stimulation pulse, and initiates a change in the neuromodulation therapy based on the measurement of skin conductivity.

### BRIEF DESCRIPTION OF THE DRAWINGS

Various aspects of apparatuses and methods will now be presented in the detailed description by way of example, and not by way of limitation, with reference to the accompanying drawings, wherein:

FIG. 1 is an illustration of a neuromodulation system including an implanted neuromodulation device and an external unit positioned relative to a body.

FIG. 2 is a block diagram of the neuromodulation system of FIG. 1.

FIG. 3 is a schematic illustration of components of the implantable neuromodulation device of FIG. 1.

FIG. 4 is another schematic illustration of components of the implantable neuromodulation device of FIG. 1.

FIG. 5 is an illustration of an example circuit architecture of components of the implantable neuromodulation device of FIG. 1.

FIG. 6 includes illustrations of stimulation pulses output by an implanted neuromodulation device based on a signal transmitted by the external unit.

FIG. 7A is a flowchart of a method of neuromodulation therapy that involves sensing and analysis of electrical potentials resulting from delivery of stimulation therapy.

FIG. 7B is a flowchart of a process for repositioning an external unit.

FIG. 8 are schematic illustrations of sensing elements of an external unit at different positions relative to stimulation elements of an implanted neuromodulation device.

FIG. 9 is a flowchart of a method of neuromodulation therapy that involves sensing and analysis of electrical signal waveforms resulting from delivery of stimulation therapy.

FIG. 10 is a flowchart of a method of measuring tissue conductivity.

FIG. 11 is a flowchart of a method of neuromodulation therapy that involves sensing an analysis of evoked compound action potentials (ECAPs) resulting from delivery of stimulation therapy.

FIG. 12 is an illustration of a signal sensed and recorded by the neuromodulation system of FIG. 1 during delivery of electrical stimulation by the system, wherein the signal exhibits a series of evoked stimulation responses (or evoked neural responses), each with a corresponding stimulation artifact and evoked compound action potential (not visible).

FIG. 13 is an illustration of a stimulation pulse (upper waveform) delivered by the neuromodulation system of FIG. 1, and a corresponding stimulation artifact (lower waveform) and evoked compound action potential (not visible).

FIG. 14 is an illustration of an evoked compound action potential waveform.

FIG. 15 is a flowchart of a method quantifying the average energy in evoked compound action potentials in real-time.

FIG. 16A is a block diagram illustration of a deconvolution based inverse filter that may be used to implement the method of FIG. 15.

FIG. 16B is a block diagram illustration of an adaptive filter that may be used to implement the method of FIG. 15.

FIG. 17 is an illustration of three consecutive cycles of stimulation artifacts and evoked compound action potentials (not visible).

FIG. 18 is an illustration of the three consecutive cycles of stimulation artifacts of FIG. 17 after filtering in accordance with the method of FIG. 15, wherein the presence of the stimulation artifacts is reduced, and the evoked compound action potentials are now visible.

FIG. 19 is an illustration of the mean squared error minima identifying the beginning of each evoked compound action potential for the three consecutive cycles of FIG. 18.

### DETAILED DESCRIPTION

Disclosed herein is a neuromodulation system having an external unit that senses electrical potentials that result from delivery of stimulation therapy by an implanted neuromodulation device, and analyzes the electrical potentials to initiate changes in neuromodulation therapy to ensure energy-efficient delivery of therapy. Also disclosed herein is a neuromodulation system having an external unit that senses electrical signal waveforms that result from delivery of stimulation therapy, and analyzes the waveforms to initiate changes in neuromodulation therapy that ensure delivery of therapy that does not activate motor control neurons.

Also disclosed herein is a neuromodulation system having an external unit that obtained measurements of skin conductivity that results from delivery of stimulation therapy by an implanted neuromodulation device, and processes the measurement of skin conductivity as a correlation of sympathetic nervous system response, and initiates changes in neuromodulation therapy to reduce sympathetic nervous system response to stimulation therapy. Also disclosed herein is a neuromodulation system having an external unit that senses evoked compound action potentials (ECAPs) resulting from delivery of stimulation therapy by an implanted neuromodulation device, and analyzes the ECAPs to initiate changes in neuromodulation therapy to ensure delivery of effective therapy, e.g., therapy that results in neural recruitment, and to ensure effective sensing of electrical activity resulting from delivery of therapy.

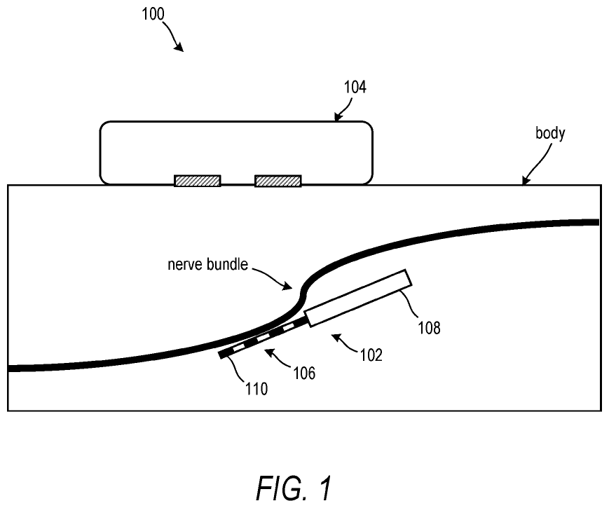

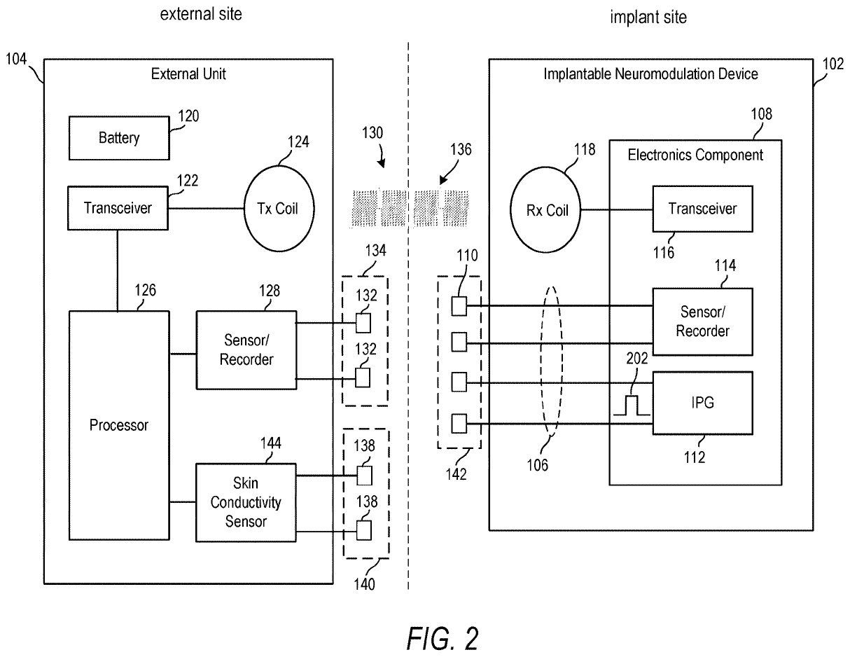

With reference to FIGS. 1 and 2, disclosed herein is a wireless neuromodulation system 100 that includes an implantable neuromodulation device 102 configured to be implanted in a body adjacent a nerve bundle, and an external unit 104 configured to provide power to the implantable neuromodulation device and transmit stimulation commands to the device.

The implantable neuromodulation device 102 includes one or more electrode platforms 106 connected to an electronics component 108. The one or more electrode platforms 106 include a number of electrodes 110. While the example implantable neuromodulation device 102 shown in FIG. 1 includes a single electrode platform 106 with four electrode 110, an implantable neuromodulation device may be configured with additional electrode platforms, and the electrode platforms may be configured with more or less than four electrodes.

The electronics component 108 includes an application specific integrated circuit (ASIC) that includes circuitry that functions as an implantable pulse generator (IPG) 112. The IPG 112 is configured to output neuromodulation therapy, e.g., electrical stimulation pulses, through the electrode platform 106. The ASIC may include additional circuitry that functions as a signal sensor/recorder 114 that captures neural activity through the electrode platform 106. The electronics component 108 also includes power/communication components, e.g., a transceiver 116 and an antenna 118 configured to communicate with the external unit 104. In an embodiment, the antenna 118 is a coil, e.g., a receive (Rx) coil. In some embodiments, the ASIC includes additional circuitry configured to harness energy and obtain therapy control information from signals transmitted by the external unit 104.

The external unit 104 includes a battery 120, power/communications components, e.g., a transceiver 122 and an antenna 124, and a processor 126. In an embodiment, the antenna 124 is a coil, e.g., a transmit (Tx) coil. In some embodiments the processor 126 is configured to control the transceiver 122 and an antenna 124 to transmit and modulate a carrier signal 130, e.g., a radio-frequency (RF) signal, in a way that provides power to the implantable neuromodulation device 102 and provides information that controls the delivery of neurostimulation by the implantable neuromodulation device 102. Details on the providing of power and control information through an RF signal may be found in International Publication Number WO 2021/055146, entitled “Wirelessly Powered Stimulator”, which is incorporated in this disclosure by reference.

The external unit 104 also includes an electrophysiology signal sensor/recorder 128, and one or more sensing elements 132, e.g., electrodes, that are electrically coupled to the electrophysiology signal sensor/recorder 128. The sensing elements 132 are configured to be placed and held in contact with tissue at an external sensing site 134 such that the sensing elements in conjunction with the electrophysiology signal sensor/recorder 128 are able to capture or sense neurological activity resulting from operation of the implantable neuromodulation device 102. For example, the sensing elements 132 may be positioned to capture or sense neural activity resulting from delivery of electrical stimulation through the electrode platform 106 of the implantable neuromodulation device 102.

In accordance with embodiments disclosed herein, signals captured or sensed at an external sensing site 134 by the sensing elements 132 in conjunction with the electrophysiology signal sensor/recorder 128 may be processed by the processor 126 to detect and analyze electrical potentials resulting from delivery of stimulation therapy by an implanted neuromodulation device, and to initiate changes in neuromodulation therapy to ensure energy-efficient delivery of therapy.

In accordance with embodiments disclosed herein, signals captured or sensed at an external sensing site 134 by the sensing elements 132 in conjunction with the electrophysiology signal sensor/recorder 128 may be processed by the processor 126 to analyze electrical signal waveforms resulting from delivery of stimulation therapy, and to initiate changes in neuromodulation therapy that ensure delivery of therapy that does not activate motor control neurons.

In accordance with embodiments disclosed herein, signals captured or sensed at an external sensing site 134 by the sensing elements 132 in conjunction with the electrophysiology signal sensor/recorder 128 may be processed by the processor 126 to detect and analyze evoked compound action potentials (ECAPs) resulting from delivery of stimulation therapy by an implanted neuromodulation device, and to initiate changes in neuromodulation therapy to ensure delivery of effective therapy, e.g., therapy that results in neural recruitment, and to ensure effective sensing of electrical activity resulting from delivery of therapy.

The external unit 104 also includes a skin conductivity sensor 144 having a pair of sensing elements 138, e.g., electrodes. The sensing elements 138 are configured to be placed and held in contact with tissue at an external site 140 such that the skin conductivity sensor 144 is able to transmit a small DC current between its sensing elements and to derive a measurement of skin conductivity therefrom.

In accordance with embodiments disclosed herein, measures of skin conductivity obtained at an external site 140 by the skin conductivity sensor 144 may be processed by the processor 126 to correlate them with sympathetic nervous system response and to initiate changes in neuromodulation therapy to reduce sympathetic nervous system response.

The processor 126 may also be configured to process neural information included in neurological activity captured or sensed by the electrode platform 106 of the implantable neuromodulation device 102 and received by the external unit 104 from the implantable neuromodulation device. To this end, the implantable neuromodulation device 102 and the external unit 104 are respectively configured to communicate through their communication components, e.g., antennas 118, 124 and transceivers 116, 122, such that neurological activity captured or sensed by the implantable neuromodulation device is transmitted to the external unit in real time.

In accordance with embodiments disclosed herein, signals captured at an implant site 142 by an electrode platform 106 in conjunction with the signal sensor/recorder 114 of the implantable neuromodulation device 102 may be transmitted to the external device 104 in real time. The signals can be processed in real time by the processor 126 to detect and analyze evoked compound action potentials (ECAPs) resulting from delivery of stimulation therapy by an implanted neuromodulation device 102, and to initiate changes in neuromodulation therapy to ensure delivery of effective therapy, e.g., therapy that results in neural recruitment, and to ensure effective sensing of electrical activity resulting from delivery of therapy.

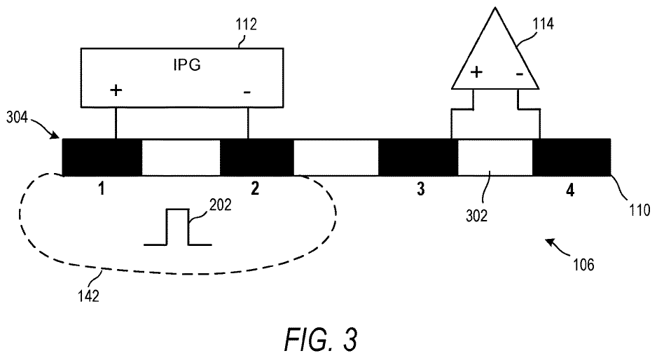

With reference to FIG. 3, in some embodiments the electrode platform 106 of the implantable neuromodulation device 102 includes an electrode-bearing body 304 having four electrodes 110. In some embodiments, the electrodes 110 are uniformly spaced apart and separated by non-conductive regions 302 of the electrode-bearing body 304. In one example configuration, the electrode-bearing body 304 is tubular shaped and 24 millimeters (mm) in length, the electrodes 110 are cylindrical and 3 mm in length, and the non-conductive regions 302 are 4 mm in length.

In the configuration shown in FIG. 3, two electrodes 110 (electrode 1 and electrode 2) are coupled to the IPG 112 of the electronics component 108 for purposes of delivering electrical stimulation pulses 202 to an implant site 142, and two electrodes 110 (electrode 3 and electrode 4) are coupled to the signal sensor/recorder 114 of the electronics component 108 for purposes of sensing electrical activity at the implant site 142. Each possible combination of two electrodes 110 can be configured in unipolar and bipolar modes. In this example, electrodes 1 and 2 are configured for bipolar electrical stimulation and electrodes 3 and 4 are configured for bipolar recording. In other configurations utilizing one electrode as a reference with the remaining electrodes configured as working electrodes, unipolar signals can be recorded, thereby enabling multiple combinations of bipolar signals to be derived.

With reference to FIGS. 2 and 3, in some embodiments the implantable neuromodulation device 102 is configured to receive radio-frequency (RF) signals 130 (also referred to as Tx signals or incident signals) from the external unit 104 and to output stimulation pulses 202 to an electrode 110 based on the RF signals. In some embodiments the RF signals are in a medical device radiocommunication (MedRadio) frequency range, e.g., 401-406, 413-419, 426-432, 438-444, and 451-457 MHz. To this end, the IPG 112 of the implantable neuromodulation device 102 may be configured to produce stimulation pulses 202 having different pulse energies based on the characteristics or configuration of the RF signal 130.

For example, as disclosed later below the characteristics or configuration of the RF signal 130 may determine the widths and/or amplitudes (and thus the pulse energy) of a stimulation pulse 202. The implantable neuromodulation device 102 is also configured to harvest energy from the RF signals 130. Details on the generation and output of stimulation pulses and energy harvesting may be found in International Publication Number WO 2021/055146, entitled “Wirelessly Powered Stimulator.” Details may also be found in “A 430-MHz Wirelessly Powered Implantable Pulse Generator With Intensity/Rate Control and Sub-1 μA Quiescent Current Consumption,” Honeming Lyu et al, IEEE Transactions on Biomedical Circuits and Systems, Vol. 13, No. 1, February 2019, which are incorporated in this disclosure by reference.

The processor 126 of the external unit 104 is configured to control the operation of the transceiver 122 to generate an RF signal 130 having a configuration that determines stimulation pulses energy and to transmit the RF signal to the implantable neuromodulation device 102. As noted above, the configuration of the RF signal 130, in turn, controls a characteristic, e.g., width and/or amplitude, of the stimulation pulse 202 produced by the IPG 112 of the implantable neuromodulation device 102. For example, with reference to FIG. 2, notches 136 can be included in the RF signal 130 to control the width of stimulation pulses 202, and to control the rate at which stimulation pulses are output by the IPG 112. A notch 136 may be included by reducing the amplitude of the RF signal 130 to a percentage of the peak amplitude that is used for purposes of energy harvesting by the implantable neuromodulation device 102.

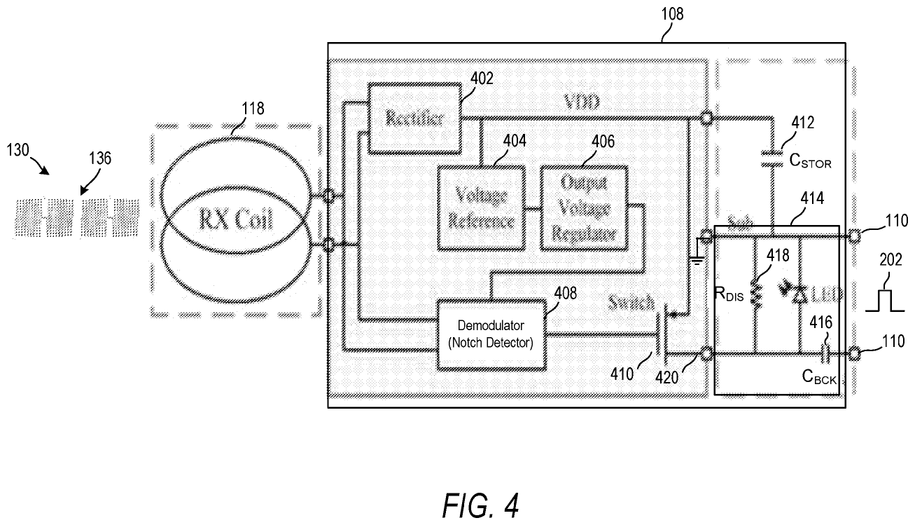

With reference to FIG. 4, in some embodiments, the electronics component 108 of the implantable neuromodulation device 102 includes a rectifier 402, a voltage reference 404, an output voltage regulator 406, a demodulator 408, a switch 410, an energy storage capacitor 412, and stimulation circuitry 414. The Rx coil 205 is configured to receive RF signals 130. The rectifier 402 is configured to rectify the RF signal 130 to generate an output voltage (VDD). Depending on the open/closed state of the switch 410, the output voltage VDD is coupled to the energy storage capacitor 412 or to the stimulation circuitry 414. When coupled to the energy storage capacitor 412, the output voltage VDD charges the energy storage capacitor.

Continuing with reference to FIG. 4, the output voltage VDD provided by the rectifier 402 is compared to a voltage reference 404 through the output voltage regulator 406. To this end, the output voltage regulator 406 is configured to compare fractions of VDD with a constant voltage reference 404. In some configurations, when the output voltage VDD exceeds a first threshold value, e.g., 19/12 of the voltage reference 404, a discharge current path (not shown in FIG. 4) is enabled to discharge excess charge from the energy storage capacitor 412. When the output voltage VDD is less than a second threshold value, e.g., 19/16 of the voltage reference 404, the output voltage regulator 406 disables the demodulator 408. Circuitry within the output voltage regulator 406 sets the first and second threshold values of the voltage reference 404 to thereby regulate the amplitude of the stimulation pulse. For example, in one configuration, the amplitude may be regulated to be in the range of 2.7 volts and 3.6 volts.

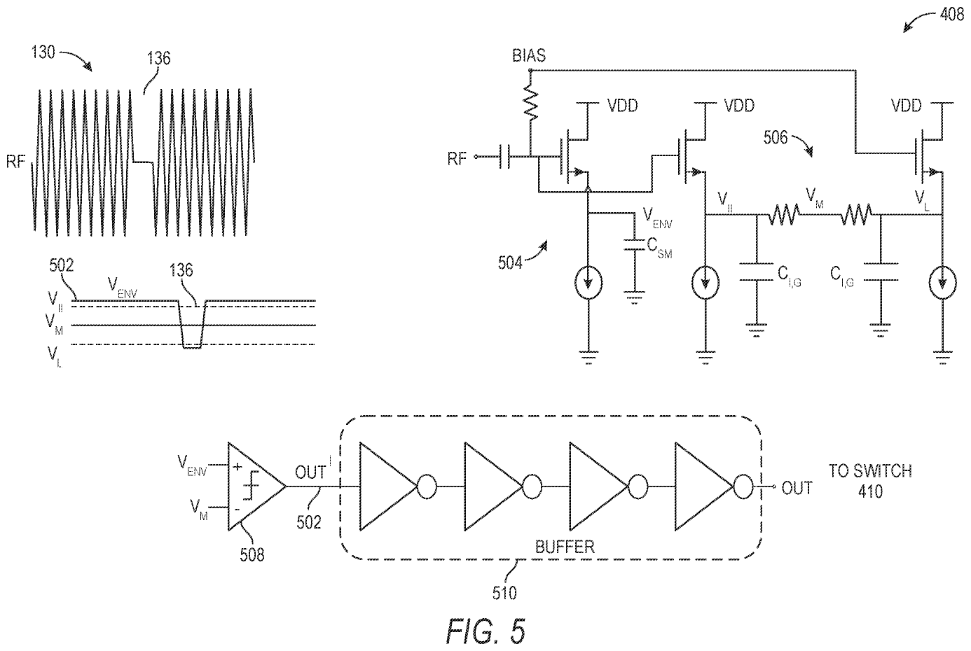

As shown in FIG. 4, the RF signals 130 are also received at the demodulator 408. The demodulator 408 is configured to process the RF signals 130 to control the on/off state of the switch 410. To this end, and with additional reference to FIG. 5, the demodulator 408 is configured to output a timing signal 502 that replicates the timing of notches 136 present in the RF signal 130. In view of this functionality, the demodulator 408 may be referred to as a notch detector. The high end, low end, and transient envelope of the RF signal 130 are denoted as V_{H}, V_{L}, and V_{ENV}, respectively in the timing signal 502. The RF signal 130 is input to circuitry 504 of the demodulator that includes a V_{ENV} detection branch, a V_{H} detection branch, and a V_{L} detection branch. The V_{ENV} detection branch may use a relatively small capacitor C_{SM} to extract V_{ENV} from the Tx signal, while V_{H} and V_{L} can be extracted on larger capacitors with and without the AC input, respectively. The average V_{M}, e.g., the average of the high end V_{H} and the low end V_{L}, can be obtained through a resistive divider 506.

The average V_{M} is input to a comparator 508 and compared with V_{ENV} to reconstruct the timing of notches 136 included in the RF signal 130. Capacitors C_{SM} and C_{WC} can be selected to be e.g., 100 fF and e.g., 36 pF, respectively. As C_{SM}<<C_{LG}, the average V_{M} can be considered as constant so that the discharging and charging of C_{SM} determines the delays from the starting point of a notch 136 and the ending point of a notch, respectively. The timing signal 502 at the output of the comparator 508 can then be sharpened by a following buffer 510 and then provided to the input of the switch 410.

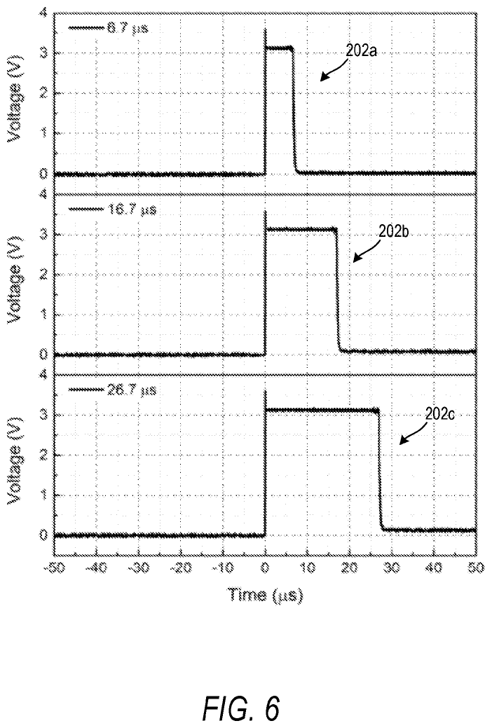

With continued reference to FIGS. 4 and 5, upon detection of a notch 136, the demodulator 408 sets the switch 410 to a closed state and holds the switch in the closed state until the notch is no longer detected. During the time the switch 410 is closed, the output voltage VDD and the energy storage capacitor 412 are coupled to the stimulation circuitry 414 and a stimulation pulse is generated and delivered through the electrodes 110. The duration of the notch 136 determines the pulse width of the stimulation pulse, and thus the pulse energy of the stimulation pulse. Examples of different stimulation pulses output by the IPG 112 of the implantable neuromodulation device 102 are shown in FIG. 6, which includes a first stimulation pulse 202a having a pulse width of 6.7 μs triggered by a 10 μs notch 136, a second stimulation pulse 202b having a pulse width of 16.7 μs triggered by a 20 μs 136, and a third stimulation pulse 202c having a pulse width of 26.7 μs triggered by a 30 μs notch 136.

Regarding the stimulation circuitry 414 of the electronics component 108, the circuitry includes a DC-block capacitor 416 and a discharge resistor 418. The DC-block capacitor 416 is coupled to the output 420 of the switch 230 and to the electrode 110. The DC-block capacitor 416 provides charge-neutralization and prevents any release of DC charge to the electrode 110. The discharge resistor 418 nulls the accumulated charge on C_{BCK}.

### Power Transmission and Stimulation Energy Control Based on Electrical Potential Detection and Analysis by an External Device

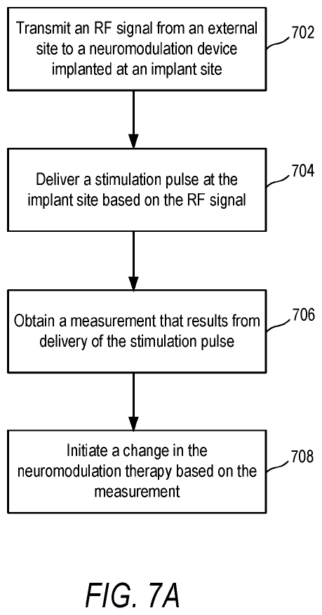

With primary reference to FIG. 7A and additional reference to FIG. 2, a neuromodulation system having an external device 104 located at an external site and a neuromodulation device 102 implanted at an implant site may be configured to enable a method of neuromodulation therapy, where a measurement resulting from delivery of stimulation therapy by the implanted neuromodulation device are sensed and analyzed by the external device to initiate changes in neuromodulation therapy to ensure energy-efficient delivery of therapy.

To this end, at block 702, the external device 104 transmits an RF signal 130 from the external site to the neuromodulation device 102 implanted at an implant site. The RF signal 130 may be transmitted by the communication components, e.g., transceiver 122 and antennas 124, of the external device 104 and received by the communication components, e.g., antenna 118 and transceiver 116, of the neuromodulation device 102 using known RF communication techniques. The RF signal 130 may be configured with notches 136 for purposes of controlling stimulation therapy as described above with reference to FIGS. 4, 5, and 6, and in International Publication Number WO 2021/055146, entitled “Wirelessly Powered Stimulator”.

At block 704, the neuromodulation device 102 delivers one or more stimulation pulses 202 at the implant site based on the RF signal 130. The stimulation pulses 202 can be delivered based on the RF signal 130 as described above with reference to FIGS. 4, 5, and 6, and in International Publication Number WO 2021/055146, entitled “Wirelessly Powered Stimulator”.

At block 706, the external device 104 obtains a measurement resulting from delivery of the stimulation pulse 202. The measurement is obtained through a sensing channel of the external device 104. The sensing channel may be a pair of electrodes 132 and sensing circuitry, such as a differential amplifier, included in the signal sensor/recorder 128. The measurement may be an electrical potential between the two electrodes 132, where the electrodes are in contact with tissue at the external sensing site 134.

In some embodiments the measurement is an individual measurement of electrical potential that results from a single stimulation pulse 202. In some embodiments the measurement is an average of individual measurements of electrical potential resulting from a corresponding number of stimulation pulse 202 deliveries. Averaging a number of measurements over time in this manner significantly improves the SNR and sensitivity of the sensing channel. In some embodiments, the measurement of electrical potential is represented by or based on an entire stimulation response (e.g., a peak voltage of the entire stimulation response). In some embodiments, the measurement of electrical potential is represented by or based on a component or feature of an entire stimulation response. For example, the measurement of electrical potential may be a measurement based on an evoked compound action potential (ECAP) component of a stimulation response.

The external device 104 is configured to obtain the measurement during a window of time following the delivery of the stimulation pulse by the neuromodulation device 102. Activation of the sensing channel of the external device 104 is synchronized with the delivery of stimulation by the neuromodulation device 102 so that sensing by the external device occurs during a window of time after a stimulation pulse is delivered. Synchronization between a sensing by the sensing channel of the external device 104 and a stimulation delivery by the neuromodulation device 102 may be based on the RF signal 130 transmitted by the external device. As described above, the RF signal 130 controls the timing of stimulation delivery. Thus, the external device 104 knows the timing of each stimulation pulse 202 and is configured to activate its sensing channel accordingly.

In some embodiments, the sensing channel may be activated so that the start of a sensing window coincides with the output of the stimulation pulse 202, or a delay time, e.g., 200 μsec, after the output of the stimulation pulse. The delay effectively blanks out the large early stimulation artifact from the sensed electrical activity. See, e.g., Chakravarthy K., FitzGerald J., Will A., Trutnau K., Corey R., Dinsmoor D., Litvak L. 2022. A Clinical Feasibility Study of Spinal Evoked Compound Action Potential Estimation Methods. Neuromodulation 2022; 25: 75-84, which is incorporated in this disclosure by reference.

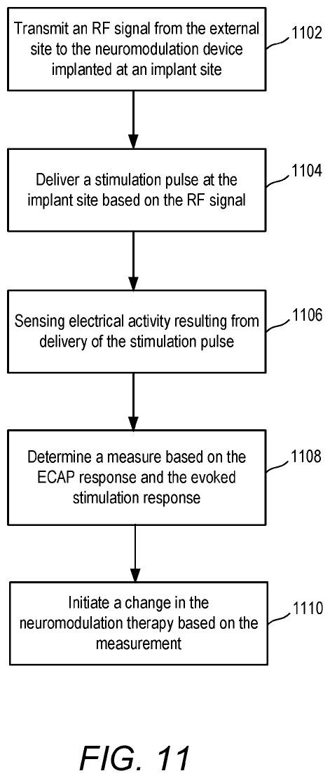

In some embodiments where the measurement is based on an ECAP, the sensing window is in the range of 1 msec to 2 msec, and the processor 126 of the external device 104 is configured to analyze electrical activity in one or more sub-windows of the sensing window to detect an ECAP. For example, a first voltage in a first sub-window between 0.3 msec and 0.6 msec may provide a first measurement of ECAP. In another example, a second voltage in a second sub-window between 0.7 msec and 1.1 msec may provide a second measurement of ECAP. In another example, the first voltage and second voltage may be processed (subtracted) to obtain a measurement of ECAP. Details on detecting ECAPS in sensed electrical activity is describe later with reference to FIGS. 12-14, and is also disclosed in International Publication Number WO 2023/211951, entitled “Systems and Methods for Closed Loop Neuromodulation”, which is incorporated in this disclosure by reference.

Continuing with reference to FIG. 7A, at block 708, the external device 104 initiates a change in the neuromodulation therapy based on the measurement of electrical potential.

In embodiments where the measurement of electrical potential measured by the sensing channel is represented by or based on an entire stimulation response (e.g., a peak voltage of the entire stimulation response), the measurement is compared to thresholds and the external device 104 initiates a change in the neuromodulation therapy based on the comparison outcome by changing the configuration of the RF signal 130 to either decrease or increase the energy of subsequent stimulation pulses 202. In some cases, in response to the measurement of electrical potential being above an upper level (e.g., above 3 millivolts) by a threshold amount (e.g., between 100 microvolts and 300 microvolts above 3 millivolts), the external device 104 initiates a change in the neuromodulation therapy by changing the configuration of the RF signal 130 to decrease the energy of subsequent stimulation pulses 202. In other cases, in response to the electrical potential being between a lower level (e.g., about 1 millivolt) and an upper level (e.g., about 3 millivolts), the external device 104 initiates a change in the neuromodulation therapy by changing the configuration of the RF signal 130 to increase the energy of subsequent stimulation pulses 202.

In embodiments where the measurement of electrical potential measured by the sensing channel is based on a component or feature of an entire stimulation response (e.g., an ECAP), the measurement is compared to thresholds and the external device 104 initiates a change in the neuromodulation therapy based on the comparison outcome by changing the configuration of the RF signal 130 to either decrease or increase the energy of subsequent stimulation pulses 202. In some cases, in response to the measurement of electrical potential of that component or feature being above an upper level (e.g., about 3 microvolts) by a threshold amount, the external device 104 initiates a change in the neuromodulation therapy by changing the configuration of the RF signal 130 to decrease the energy of subsequent stimulation pulses 202. In other cases, in response to the measurement of electrical potential of that component or feature being between a lower level (e.g., about 1 microvolts) and an upper level (e.g., about 3 microvolts), the external device 104 initiates a change in the neuromodulation therapy by changing the configuration of the RF signal 130 to increase the energy of subsequent stimulation pulses 202

In either embodiment, as described above the RF signal 130 may include a notch 136 having a duration, and changing the configuration of the RF signal to change the energy of subsequent stimulation pulses 202 comprises changing the duration. To this end, changing the configuration of the RF signal to decrease the energy of subsequent stimulation pulses 202 comprises reducing the duration, while changing the configuration of the RF signal to increase the energy of subsequent stimulation pulses 202 comprises increasing the duration. In some embodiments, the duration of the notch 136 is incrementally reduced until the measurement exceeds the upper level by an amount less than the threshold amount. In some embodiments, the duration of the notch 136 is incrementally increased until the measurement of electrical potential exceeds the upper level by an amount less than the threshold amount.

With reference to FIG. 7B, in some embodiments, initiating a change in the neuromodulation therapy comprises, at block 710, initiating a process to reposition the external device 104 in response to the measurement of electrical potential measured by the sensing channel being less than a threshold level, e.g., the lower level (e.g., about 1 millivolt) referenced above. For example, the external device 104 may be configured to communicate with a user device, e.g., a mobile phone, and to provide a sound or visual guide that helps the user find the optimal location for the external unit. The external device 104 is repositioned by placing the pair of electrodes 132 of the sensing channel at a different external sensing site 134.

At block 712, the external device transmits an RF signal 130 from the different external sensing site 134 to the neuromodulation device 102 implanted at an implant site, after the user repositions the external device 104. The RF signal 130 may be transmitted by the communication components, e.g., transceiver 122 and antennas 124, of the external device 104 and received by the communication components, e.g., antenna 118 and transceiver 116, of the neuromodulation device 102 using known RF communication techniques. The RF signal 130 may be configured with notches 136 for purposes of controlling stimulation therapy as described above with reference to FIGS. 4, 5, and 6, and in International Publication Number WO 2021/055146, entitled “Wirelessly Powered Stimulator”.

At block 714, the neuromodulation device 102 delivers a stimulation pulse 202 at the implant site based on the RF signal 130. The stimulation pulses 202 can be delivered based on the RF signal 130 as described above with reference to FIGS. 4, 5, and 6, and in International Publication Number WO 2021/055146, entitled “Wirelessly Powered Stimulator”.

At block 716, the external device 104 obtains a measurement resulting from delivery of the stimulation pulse. The measurement is obtained through a sensing channel of the external device 104. The sensing channel includes the pair of repositioned electrodes 132 and sensing circuitry, such as a differential amplifier, included in the signal sensor/recorder 128. The measurement may be an electrical potential between the two electrodes 132, where the electrodes are in contact with tissue at the external sensing site 134.

At block 718, the external device 104 determines if the measurement of electrical potential is still less than the threshold level, e.g., lower level (e.g., about 1 millivolt). If the measurement of electrical potential is less than the threshold level, the process proceeds to block 720, where the external device 104 continues the repositioning process by providing a sound or visual guide that helps the user find the optimal location for the external unit, and returning to repeat blocks 712, 714 and 716. Changes in the measurement of electrical potential may invoke corresponding changes in the sound or visual guide. For example, if the measurement of electrical potential is closer to the threshold than it was before, the sound may be louder, or the visual guide may get brighter or may blink faster. Conversely, if the measurement of electrical potential is further from the threshold than it was before, the sound may be quieter, or the visual guide may get lighter or may blink slower.

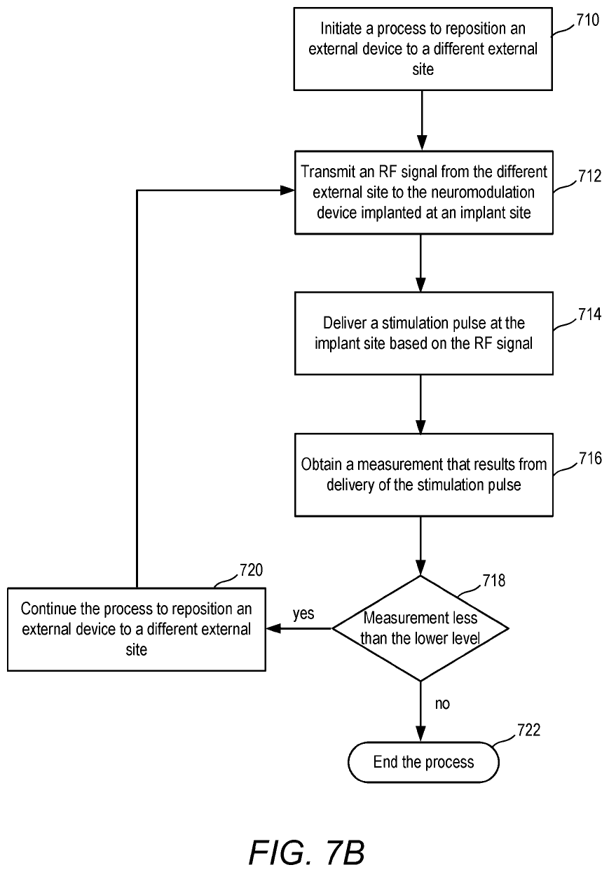

With reference to FIG. 8, the intent of the repositioning process is to place the sensing electrodes 132 of the external device 104 at a location relative to the stimulation electrodes 110 of the implanted neuromodulation device 102 that improves the sensing capability of the external device. For example, in the relative placement of panel A, the sensing electrodes 132 are positioned too far away from the stimulation electrodes 110 to effectively sense electrical activity resulting from the delivery of a stimulation pulse through the stimulation electrodes. In panel B, however, after repositioning of the sensing electrodes 132 to a location closer to, or more aligned with the stimulation electrodes 110, the sensing electrodes are able to effectively sense electrical activity resulting from the delivery of a stimulation pulse through the stimulation electrodes 110.

Returning to block 718, if the measurement of electrical potential is not less than the threshold level, e.g., the lower level (e.g., about 1 millivolt), the process proceeds to block 722 where the external device 104 ends the repositioning process.

### Power Transmission and Stimulation Energy Control Based on Detection of Motor Neuron Activation by an External Device

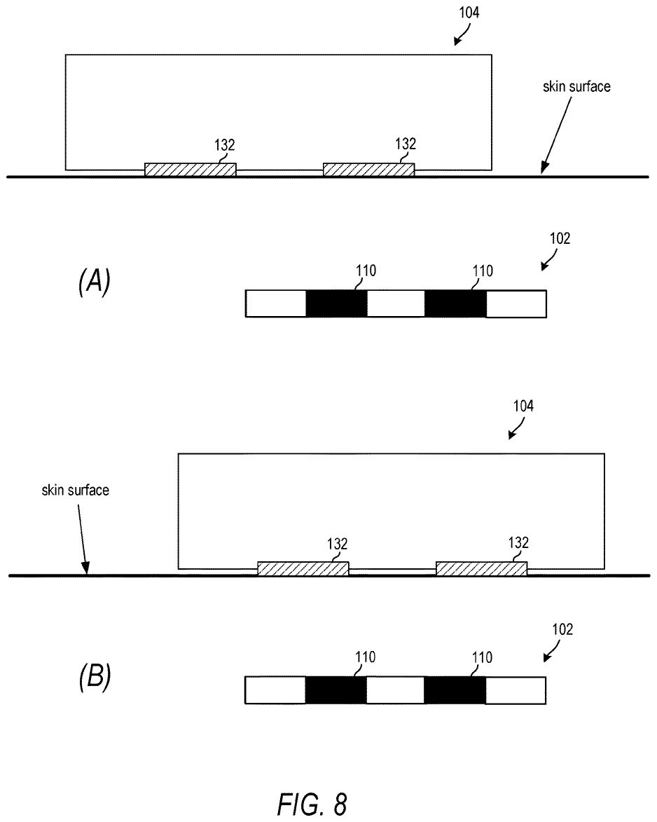

With primary reference to FIG. 9 and additional reference to FIG. 2, a neuromodulation system having an external device 104 located at an external sensing site 134 and a neuromodulation device 102 implanted at an implant site may be configured to enable a method of neuromodulation therapy, where electrical signal waveforms resulting from delivery of stimulation therapy are analyzed by the external device 104 to initiate changes in neuromodulation therapy that ensure delivery of therapy that does not activate motor control neurons.

To this end, at block 902, the external device 104 transmits an RF signal 130 from the external site to the neuromodulation device 102 implanted at an implant site. The RF signal 130 may be transmitted by the communication components, e.g., transceiver 122 and antennas 124, of the external device 104 and received by the communication components, e.g., antenna 118 and transceiver 116, of the neuromodulation device 102 using known RF communication techniques. The RF signal 130 may be configured with notches 136 for purposes of controlling stimulation therapy as described above with reference to FIGS. 4, 5, and 6, and in International Publication Number WO 2021/055146, entitled “Wirelessly Powered Stimulator”.

At block 904, the neuromodulation device 102 delivers one or more stimulation pulse 202 at the implant site based on the RF signal 130. The stimulation pulses 202 can be delivered based on the RF signal 130 as described above with reference to FIGS. 4, 5, and 6, and in International Publication Number WO 2021/055146, entitled “Wirelessly Powered Stimulator”.

At block 906, the external device 104 obtains a signal waveform representing neural activity resulting from delivery of the stimulation pulse 202. The signal waveform is obtained through a sensing channel of the external device 104. The sensing channel may be a pair of electrodes 132 and sensing circuitry, such as a differential amplifier, included in the signal sensor/recorder 128. The signal waveform may be a stimulation response 1200 waveform that includes an evoked compound action potential (ECAP) 1208, as described later with reference to FIGS. 12-14.

In some embodiments the signal waveform is an individual signal waveform of a stimulation response 1200 that results from a single stimulation pulse 202. In some embodiments the signal waveform is an average of individual signal waveforms resulting from a corresponding number of stimulation pulse 202 deliveries. Averaging a number of signal waveforms over time in this manner significantly improves the SNR and sensitivity of the sensing channel.

The external device 104 is configured to obtain a signal waveform during a window of time following the delivery of each stimulation pulse 202 by the neuromodulation device 102. Activation of the sensing channel of the external device 104 is synchronized with the delivery of stimulation by the neuromodulation device so that sensing by the external device occurs during a window of time after a stimulation pulse is delivered. Synchronization between sensing by the sensing channel of the external device 104 and stimulation delivery by the neuromodulation device 102 may be based on the RF signal 130 transmitted by the external device. As described above, the RF signal 130 controls the timing of stimulation delivery. Thus, the external device 104 knows the timing of each stimulation pulse and is configured to activate its sensing channel accordingly.

In some embodiments, the sensing channel may be activated so that the start of a sensing window coincides with the output of the stimulation pulse, or a delay time, e.g., 200 μsec, after the output of the stimulation pulse. The delay effectively blanks out the large early stimulation artifact from the sensed electrical activity. See, e.g., Chakravarthy K., FitzGerald J., Will A., Trutnau K., Corey R., Dinsmoor D., Litvak L. 2022. A Clinical Feasibility Study of Spinal Evoked Compound Action Potential Estimation Methods. Neuromodulation 2022; 25: 75-84, which is incorporated in this disclosure by reference.

In some embodiments, the sensing window is in the range of 1 msec to 2 msec, and the processor 126 of the external device 104 is configured to analyze a signal waveform (corresponding to either an individual waveform or an average waveform derived from a plurality of individual waveforms) of electrical activity in one or more sub-windows of the sensing window to detect an ECAP. For example, a voltage minimum in a first sub-window between 0.3 msec and 0.6 msec may provide a measurement of ECAP. In another example, a voltage maximum in a second sub-window between 0.7 msec and 1.1 msec may provide a measurement of ECAP. In another example, the minimum voltage and maximum voltage may be processed (subtracted) to obtain a measurement of ECAP. Details on detecting ECAPS in sensed electrical activity is describe later with reference to FIGS. 12-14, and is also disclosed in International Publication Number WO 2023/211951, entitled “Systems and Methods for Closed Loop Neuromodulation”, which is incorporated in this disclosure by reference.

Continuing with reference to FIG. 9, at block 908, the external device 104 initiates a change in the neuromodulation therapy based on the signal waveform. To this end, the external device 104 is configured to process the signal waveform to determine if motor control neurons were activated by the stimulation pulse. For example, the processor 126 of the external device 104 may be configured to process the morphology of the signal waveform to identify features (or signatures) that are present when motor control neurons are activated.

In response to determining motor control neurons were activated by the stimulation pulse based on the signal waveform, the external device 104 changes the configuration of the RF signal 130 to decrease the energy of subsequent stimulation pulses 202. In some embodiments, the RF signal 130 comprises a notch 136 having a duration, and changing the configuration of the RF signal to decrease the energy of subsequent stimulation pulses 202 comprises reducing the duration. In some embodiments, the duration of the notch 136 is incrementally reduced until the motor control neurons are not activated by a subsequent stimulation pulse 202.

### Power Transmission and Stimulation Energy Control Based on Skin Conductivity Analysis by an External Device

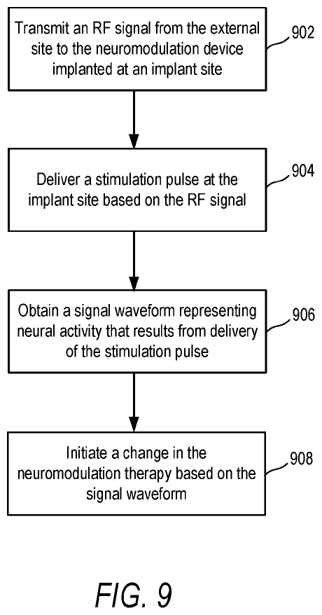

With primary reference to FIG. 10 and additional reference to FIG. 2, a neuromodulation system having an external device 104 located at an external site 140 and a neuromodulation device 102 implanted at an implant site may be configured to enable a method of neuromodulation therapy, where measures of skin conductivity during delivery of stimulation therapy by the implanted neuromodulation device are obtained by the external device and analyzed to adjust stimulation energy.

To this end, at block 1002, the external device 104 transmits an RF signal 130 from the external site to the neuromodulation device 102 implanted at an implant site. The RF signal 130 may be transmitted by the communication components, e.g., transceiver 122 and antennas 124, of the external device 104 and received by the communication components, e.g., antenna 118 and transceiver 116, of the neuromodulation device 102 using known RF communication techniques. The RF signal 130 may be configured with notches 136 for purposes of controlling stimulation therapy as described above with reference to FIGS. 4, 5, and 6, and in International Publication Number WO 2021/055146, entitled “Wirelessly Powered Stimulator”.

At block 1004, the neuromodulation device 102 delivers a stimulation pulse at the implant site based on the RF signal 130. The stimulation pulses 202 can be delivered based on the RF signal 130 as described above with reference to FIGS. 4, 5, and 6, and in International Publication Number WO 2021/055146, entitled “Wirelessly Powered Stimulator”.

At block 1006, the external device 104 obtains a measurement of skin conductivity through a skin-conductivity sensor 144. The skin-conductivity sensor 144 may be a galvanic skin response (CSR) device, e.g., capacitive, resistive, piezoelectric, and thermocouple, coupled to a pair of closely spaced electrodes 138 on the external device 104. The measurement of skin conductivity may be obtained, for example, by inducing a DC current between the pair of electrodes 138 while the electrodes are in contact with tissue at the external site 140. The measurement may correspond to a measurement of perspiration from the gland that correlates with sympathetic response.

In some embodiments the measurement of skin conductivity is an individual measurement of skin conductivity that results from a single stimulation pulse 202. In some embodiments the measurement of skin conductivity is an average of individual measures of skin conductivity that result from a corresponding number of stimulation pulses 202. Averaging a number of measures of skin conductivity over time in this manner significantly improves the SNR and sensitivity of the skin-conductivity sensor.

The external device 104 may be configured to obtain the measurement of skin conductivity following the delivery of the stimulation pulse 202 by the neuromodulation device 102. Activation of the skin-conductivity sensor 144 of the external device 104 is synchronized with the delivery of stimulation by the neuromodulation device 102 so that sensing by the external device occurs during a window of time after a stimulation pulse 202 is delivered. Synchronization between sensing by the skin-conductivity sensor 144 of the external device 104 and the stimulation delivery by the neuromodulation device 102 may be based on the RF signal 130 transmitted by the external device. As described above, the RF signal 130 controls the timing of stimulation delivery. Thus, the external device 104 knows the timing of each stimulation pulse 202 and is configured to activate the skin-conductivity sensor 144 accordingly.

Continuing with reference to FIG. 10, at block 1008, the external device 104 initiates a change in the neuromodulation therapy based on the measurement of skin conductivity at the external site 140. To this end, the external device 104 is configured to process the measurement of skin conductivity to correlate it with sympathetic response. For example, the measurement of skin conductivity varies as a function of skin perspiration, and perspiration increase correlates with an increase sympathetic response. Accordingly, the external device 104 may monitor for a measurement of skin conductivity obtained during neurostimulation therapy that is above a threshold and respond by modifying the neurostimulation therapy to reduce the sympathetic response. The measurement of skin conductivity may be a measure of electrodermal activity (EDA) obtained during neurostimulation therapy and the threshold may be a percentage increase in EDA measured during neurostimulation therapy relative to EDA measured in the absence of neurostimulation therapy.

In response to determining the measurement of skin conductivity is above the threshold, the external device 104 changes the configuration of the RF signal 130 to decrease the energy of subsequent stimulation pulses 202. In some embodiments, the RF signal 130 comprises a notch 136 having a duration, and changing the configuration of the RF signal to decrease the energy of subsequent stimulation pulses 202 comprises reducing the duration. In some embodiments, the duration of the notch 136 is incrementally reduced until the measurement of skin conductivity is no longer above the threshold.

### Power Transmission and Stimulation Energy Control Based on ECAP Detection And Analysis by External Device

Neurostimulation therapies are often limited due to a loss of therapeutic effect over time. Habituation and desensitization with increasing tolerance to the neurostimulation therapy is a considerable challenge in the treatment of disorders such as chronic pain and urinary dysfunction. This effect is exacerbated with the use of static stimulation waveforms. By applying multiple stimulation waveforms or a more stochastic dynamic pattern of waveform, the loss of therapeutic benefit can be delayed or potentially eliminated. However, to quantify the effect of stimulation some form of feedback is necessary to determine if sufficient neural recruitment of target neural fibers has been achieved. For example, neural recruitment may be evidenced by an evoked stimulation response (also referred to herein as a neural response) sensed at or near the target neural fibers and captured by a neuromodulation device. Failure to recruit the target neural fibers will provide no therapeutic benefits, whereas exceeding the upper limits of the therapeutic window can increase the risk of tissue damage over time and can cause pain. Therapeutic window (or treatment window), as used herein generally means the range between the perception by a patient of paresthesia sensation onset and discomfort from stimulation.

The current state of the art in peripheral nerve stimulation (PNS) to treat a range of chronic disorders, such as neuropathic pain and urinary disfunction, adopt open loop modalities, wherein the pulse generator output is a fixed waveform. While this approach is sufficient to provide therapeutic benefits, it is far from optimal, with adjustments to waveform amplitude performed by the patient. Variations in the electrical field at the nerve interface where the charge is delivered to a peripheral nerve fiber are common. Variations can be a result of changes in interface impedance, electrode movement and other interfering factors. Electrode movement can result from cardiac and respiratory motion or ambulation. This can impact the ability of the device to elicit an action potential (AP) response or provide the desired stimulation response consistently. Therefore, by dynamically adjusting stimulation doses based on feedback, consistent neural recruitment is possible. Stimulation dose, as used herein means the type and/or pattern of stimulation that is delivered in order to evoke a neural response at the target fibers. The type of stimulation may be, for example, electrical pulse waveform stimulation, where a pulse is defined by amplitude and pulse width. The pattern of stimulation may be a single pulse, a pulse train (a series of stimulation pulses separated by a brief time interval specified by a frequency parameter) or continuous stimulation for a period of time.

A means of stimulation feedback is highly desirable to optimize therapy delivery. Fundamentally, feedback to simply acknowledge neural recruitment could have significant benefits. One of the failures of occipital nerve stimulation (ONS) to treat migraine has been due to lead movement and migration resulting in a failure to recruit target neural fibers, while patients believed they were receiving therapy. Moreover, the introduction of new stimulation waveforms that stimulate at sub-paresthesia levels complicate open loop systems further, where it becomes increasingly more difficult for a patient to adjust their stimulation therapy effectively.

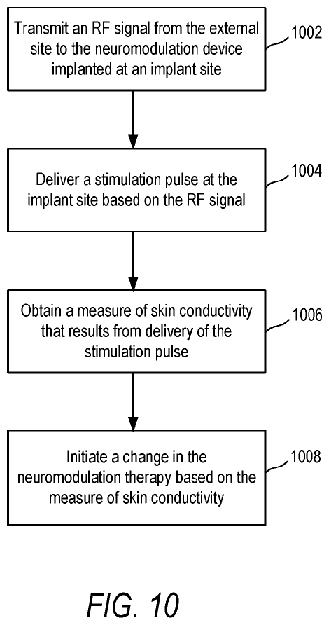

With primary reference to FIG. 11 and additional reference to FIG. 2, a neuromodulation system having an external device 104 located at an external site and a neuromodulation device 102 implanted at an implant site may be configured to enable a method of neuromodulation therapy, where evoked compound action potentials (ECAPs) resulting from delivery of stimulation therapy by the implanted neuromodulation device are detected and analyzed by the external device to initiate changes in neuromodulation therapy to ensure delivery of effective therapy, e.g., therapy that results in neural recruitment, and to ensure effective sensing of electrical activity resulting from delivery of therapy.

To this end, at block 1102, the external device 104 transmits an RF signal 130 from the external site to the neuromodulation device 102 implanted at an implant site. The RF signal 130 may be transmitted by the communication components, e.g., transceiver 122 and antennas 124, of the external device 104 and received by the communication components, e.g., antenna 118 and transceiver 116, of the neuromodulation device 102 using known RF communication techniques. The RF signal 130 may be configured with notches 136 for purposes of controlling stimulation therapy as described above with reference to FIGS. 4, 5, and 6, and in International Publication Number WO 2021/055146, entitled “Wirelessly Powered Stimulator”.

At block 1104, the neuromodulation device 102 delivers a stimulation pulse at the implant site based on the RF signal 130. The stimulation pulses 202 can be delivered based on the RF signal 130 as described above with reference to FIGS. 4, 5, and 6, and in International Publication Number WO 2021/055146, entitled “Wirelessly Powered Stimulator”.

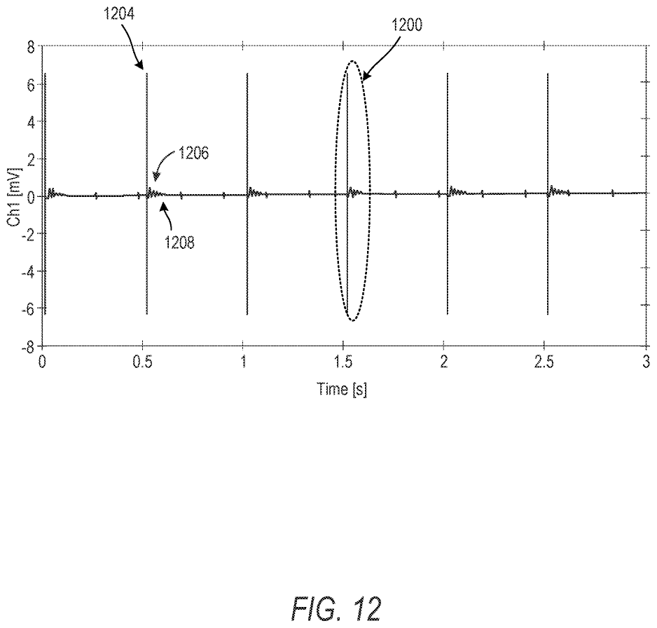

At block 1106, the external device 104 captures or senses electrical activity of the tissue resulting from delivery of the stimulation pulse. The captured or sensed electrical activity is obtained through a sensing channel of the external device 104. The sensing channel may be a pair of electrodes 132 and sensing circuitry, such as a differential amplifier, included in the signal sensor/recorder 128. With reference to FIGS. 12 and 13, the sensed electrical activity includes a stimulation response 1200 comprising an evoked stimulation response 1204, a stimulation artifact 1206, and an ECAP response 1208. The stimulation response 1200 may be represented by a waveform of electrical potential as a function of time.

In some embodiments the stimulation response 1200 is an individual response that results from a single stimulation pulse 202. In some embodiments the stimulation response 1200 is an average of individual responses that result from a corresponding number of stimulation pulse 202 deliveries. Averaging a number of stimulation responses 1200 over time in this manner significantly improves the SNR and sensitivity of the sensing channel and enables the measuring of the ECAP response 1208.

The external device 104 is configured to sense electrical activity during a window of time following the delivery of the stimulation pulse 202 by the neuromodulation device 102. Activation of the sensing channel of the external device 104 is synchronized with the delivery of stimulation by the neuromodulation device 102 so that sensing by the external device occurs during a window of time after a stimulation pulse is delivered. Synchronization between sensing by the sensing channel of the external device 104 and stimulation delivery by the neuromodulation device 102 may be based on the RF signal 130 transmitted by the external device. As described above, the RF signal 130 controls the timing of stimulation delivery. Thus, the external device 104 knows the timing of each stimulation pulse 202 and is configured to activate its sensing channel accordingly.

In some embodiments, the sensing channel may be activated so that the start of a sensing window coincides with the output of the stimulation pulse, or a delay time, e.g., 200 μsec, after the output of the stimulation pulse. The delay effectively blanks out the large early stimulation artifact from the sensed electrical activity. See, e.g., Chakravarthy K., FitzGerald J., Will A., Trutnau K., Corey R., Dinsmoor D., Litvak L. 2022. A Clinical Feasibility Study of Spinal Evoked Compound Action Potential Estimation Methods. Neuromodulation 2022; 25: 75-84, which is incorporated in this disclosure by reference.

Continuing with reference to FIG. 11, at block 1108, the external device 104 determines a measurement based on the ECAP response 1208 and the evoked stimulation response 1204. In some embodiments, the measurement is a relationship, e.g., a ratio, of the ECAP response 1208 and the evoked stimulation response 1204. For example, amplitudes of the ECAP response 1208 and the evoked stimulation response 1204 may be obtained and the measurement may be a ratio of these amplitudes. To this end, an amplitude of the ECAP response 1208 can be obtained by applying a filter to the stimulation response 1204, locating the ECAP response in the filtered stimulation response, and deriving the amplitude from a waveform corresponding to the ECAP response. The ECAP response can be located relative to the evoked stimulation response based on a known time offset. The known time offset can be determined based on previously sensed electrical activity of tissue resulting from prior deliveries of the neuromodulation therapy.

At block 1110, the external device 104 initiates a change in the neuromodulation therapy based on the measurement.

In some embodiments, in response to the measurement being above a first threshold (e.g., ECAP response/evoked stimulation response ratio of about 0.02), the external device 104 initiates a change in the neuromodulation therapy by changing the configuration of the RF signal 130 to decrease the energy of a subsequent stimulation pulse. For example, the RF signal 130 may include a notch 136 having a duration, and the configuration of the RF signal is changed to decrease the energy of the subsequent stimulation pulse by reducing the duration. In an embodiment, the duration of the notch 136 is incrementally reduced until the measurement is above the first threshold.

In some embodiments, in response to the measurement being below a second threshold (e.g., ECAP response/evoked stimulation response ratio of about 0.005), the external device 104 initiates a change in the neuromodulation therapy by changing the configuration of the RF signal 130 to increase the energy of a subsequent stimulation pulse. For example, the RF signal 130 may include a notch 136 having a duration, and the configuration of the RF signal is changed to increase the energy of the subsequent stimulation pulse by increasing the duration. In an embodiment, the duration of the notch 136 is incrementally increased until the measurement is above the second threshold.

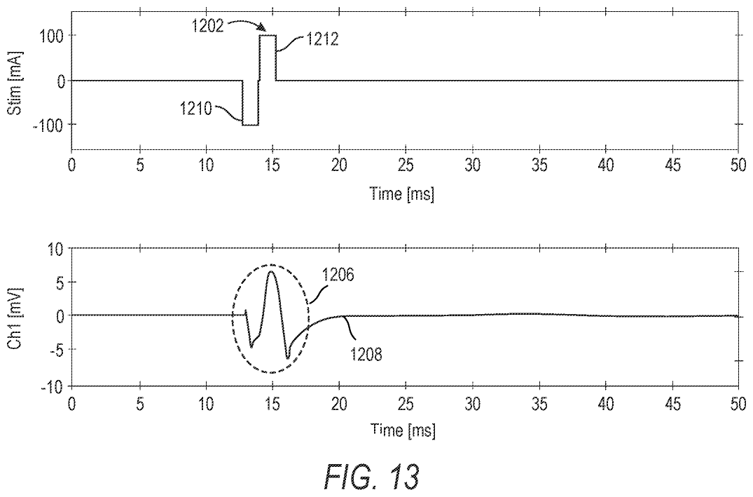

Regarding ECAPs, with reference to FIGS. 12, 13, and 14, wherein FIG. 12 illustrates a series of six stimulation responses, FIG. 13 illustrates one cathodic stimulation cycle with a zoomed in portion of its corresponding stimulation response, and FIG. 14 illustrates a typical AP response 1208 elicited through stimulation, i.e., upon delivery of an electrical stimulation pulse 1202 to a nerve bundle.

With reference to FIG. 12, each of six stimulation responses 1200 resulting from electrical activity sensed at the nerve bundle exhibits an evoked stimulation response 1204, a resultant stimulation artifact 1206, and an evoked compound action potential (ECAP) response 1208 (not visible in FIG. 12 or 13, but shown in FIG. 14). Action potential (AP) responses 1208 occur uniformly, potentially under cardiac autonomic control. The significant difference in stimulation artifact 1206 and AP response 1208 amplitude is salient.

With reference to FIG. 13, a stimulation artifact 1206 is evident from both a negative stimulus pulse 1210 and a positive charge recovery pulse 1212. In the example of FIG. 13 an AP response 1208 is generated at 20 ms, however, it is not identifiable in this example as the AP response is in the μV range and stimulation artifact 1206 occupies the mV range. The AP response 1208 occurs at 20 ms which is 7 ms from the onset of the electrical stimulation pulse 1202. Therefore, the AP is propagating at ˜2 m/s which is common for Aδ or C fibers which require 7 ms to travel the 14 mm between electrodes 110 as shown in FIG. 3.

With reference to FIG. 14, the typical AP response 1208 elicited through stimulation is measured during a period with minimal artifact and noise. In this example the AP response 1208 waveform is ˜60 μV peak-to-peak which is considerably lower amplitude than the stimulation artifact 1206 shown in FIG. 13.

Because the stimulation artifact 1206 is proportional to the charge delivered, which is dependent on the pulse amplitude and width of the electrical stimulation pulse 1202, the stimulation artifact generated at increased amplitude can be predicted once the stimulation artifact characteristics recorded on the sensing electrodes 110 are determined and modeled. This is crucial as any stimulation artifact 1206 removal routine that is adapted in the presence of neural signals will likely attenuate or remove the neural components, e.g., the AP response 1208, of interest.

In accordance with embodiments disclosed herein, a stimulation response signal, such as illustrated in FIG. 12, is processed to attenuate, or reduce the presence of stimulation artifacts 1206 without attenuating the AP response 1208. Once stimulation artifacts 1206 are attenuated, the remaining filtered signal is processed to locate the AP response 1208. As the stimulation (i.e., the delivery of electrical stimulation pulse 1202) is periodic, once the AP response 1208 to such stimulation is identified, the exact time lag between the evoked stimulation response 1204 and the AP response 1208 can be measured and used to predict the location of the AP response 1208 after the delivery of each electrical stimulation pulse 1202 (or after the detection of the evoked stimulation response 1204).

Knowing the location or time of occurrence of the AP response 1208 relative to the time of evoked stimulation response 1204 is beneficial because signal processing can be applied to that particular time segment (the segment containing the AP response 1208). The AP response 1208 may be used to quantify the overall neural response to the electrical stimulation pulse 1202. For example, if the electrical stimulation pulse 1202 is too low there will not be a neural response. In other words, there would be no stimulation response 1200 as shown in FIG. 12. As another example, if the electrical stimulation pulse 1202 is too high, a high amplitude evoked stimulation response 1204 within the stimulation response 1200 may be noted, which may be outside of the treatment window. As previously mentioned, treatment window generally means the range between the perception by a patient of paresthesia sensation onset and discomfort from stimulation. In the case of a high amplitude evoked stimulation response 1204 outside the treatment window, the patient would experience discomfort.

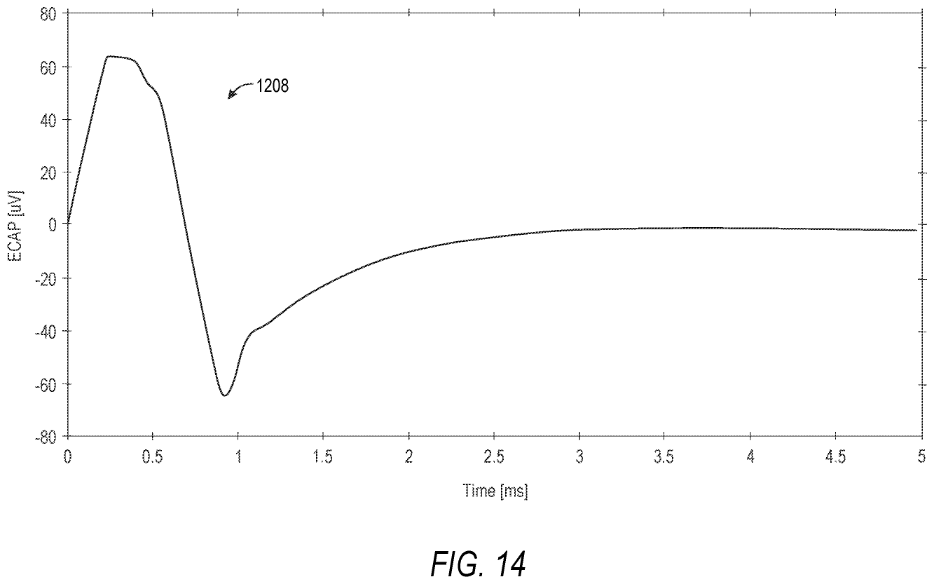

With primary reference to FIG. 15 and additional reference to FIG. 2, a method of quantifying the average energy in evoked compound action potentials (ECAPs) in a signal representing sensed electrical activity of a nerve is now described. The method involves reducing the presence of stimulation artifact 1206 in a signal representing sensed electrical activity of a nerve. The method may be performed by the external unit 104, where the external unit is configured to acquire the signal representing sensed electrical activity of a nerve. For example, the signal may be acquired through a sensing channel of the external device 104. The sensing channel may be a pair of electrodes 132 and sensing circuitry, such as a differential amplifier, included in the signal sensor/recorder 128. Alternatively, the signal may be acquired from an implantable neuromodulation device 102 that is configured to acquire the signal representing sensed electrical activity of a nerve and to transmit the signal to the external unit 104.

At block 1500, an implanted neuromodulation device 102 is activated to output stimulation therapy in the form of electrical stimulation pulses 1202. For example, when treating pain, a patient may switch the external unit 104 on when pain is excessive. The external unit 104, in turn, transmits an RF signal 130 to the implanted neuromodulation device 102 that causes the device to deliver a neuromodulation therapy to the patient. The neuromodulation therapy includes one or more electrical stimulation pulses 1202 defined by stimulation parameters e.g., pulse width, pulse amplitude, etc. Because the external unit 104 controls the delivery of the neuromodulation therapy, the stimulation parameters are known to the external unit 104.

At block 1501, the current parameters of the electrical stimulation pulses 1202 are processed by the external unit 104 to determine if a stimulation level provided by the stimulation parameters is below a known activation threshold that elicits an AP response 1208. To this end, the external unit 104 may calculate an energy level for the electrical stimulation pulses 1202 based on the parameters of the pulse. The known activation threshold is predefined and typically determined for a patient during a clinical programming session. The activation threshold may be adjusted during follow-up clinical sessions.

If the stimulation level provided by the stimulation parameters of the electrical stimulation pulse 1202 is not below the activation threshold (in other words, it is above the activation threshold and thus elicits an ECAP or AP response 1208), the process enters a filtering operation or filtering mode. To this end, the process proceeds to block 1511, where a signal representing sensed electrical activity of a nerve and acquired by the external unit 104 is processed by an adaptive filter. The acquired signal representing sensed electrical activity of a nerve may correspond to a signal like the one shown in FIG. 13.

The main purpose of the filtering operation (block 1511) is to significantly attenuate the stimulation artifact 1206 generated through stimulation. The ECAP signals or AP responses 1208 can be better derived if they are separated from the stimulation artifact 1206 that is typically orders of magnitude greater than the physiological signal of interest, as illustrated in FIG. 12. In one approach, a model is used to predict the location (time of appearance) of a stimulation artifact 1206 in the recorded signal and perform a linear subtraction to thereby significantly attenuate or remove the stimulation artifact from the recorded signal.

As illustrated in FIG. 15, when an electrical stimulation pulse 1202 is applied (block 1500) and is not below a threshold (block 1501) that would elicit a physiological response i.e., an AP response 1208, the stimulation artifact 1206 generated by a particular applied electrical stimulation pulse 1202 having a pulse waveform is measured by the sensing channel of the external unit. The medium (e.g., neural fiber) effects between the stimulating electrodes (electrodes 1 and 2 in FIG. 3) of the implanted neuromodulation device 102 and the electrodes 132 of the sensing channel of the external unit is modeled. A typical filter that preforms this modeling in real-time is an adaptive filter where the pulse waveform of the electrical stimulation pulse 1202 delivered by the stimulating electrodes (electrodes 1 and 2 in FIG. 3) is the input signal and the sensed signal sensed by the sensing channel of the external unit 104 is the reference signal.

The adaptive filter is one where the filter weights are adjusted in real-time to minimize the error between the input signals (the pulse waveform of the electrical stimulation pulse 1202) and reference signals as will be described below. If for example the adaptive filter has an FIR structure, then it can be described as follows where N is the filter model order:

$$y [ k ] = {\sum }_{n = 0}^{N - 1} W [ n ] x [ k - n ]$$

At block 1512, the filtered signal output by the adaptive filer is processed to detect AP responses 1208, where an AP response signal is detected for each stimulation response 1200. The AP response 1208 may corresponds to a signal like the one shown in FIG. 14. Several methods can be employed to detect the AP response 1208. Given the fast conduction of AP response 1208 on most neural fibers, the response will appear during stimulus. The AP response 1208 can be measured in a time window with a fixed duration from the onset of an electrical stimulation pulse 1202. Here the peak can simply be measured and used for titration on the next cycle. Methods such as cross-correlation techniques can be used to detect the AP response 1208 waveform or spike, or simple threshold techniques. However, sufficient stimulation artifact 1206 should be attenuated where the AP response 1208 will be of greater amplitude than the stimulation artifact enabling a simple threshold detection approach.

At block 1513, the detected AP responses 1208 detected are processed by the external unit 104 to derive an average AP response signal using known waveform averaging techniques. If sufficient stimulation artifact 1206 is removed the AP response 1208 will be clear in the average waveform calculated in block 1513. The average AP response signal may be used to derive a measurement relative to the evoked stimulation response 1204. For example, a measurement of relative amplitude between the average AP response signal and an average evoked stimulation response 1204 may be a basis for adjusting or changing one or more stimulation parameters, e.g., a different pulse-width, duty cycle or stimulation frequency of the stimulation therapy.

At block 1514, a determination is made to adjust one or more of the stimulation parameters defining the electrical stimulation pulses 1202 based on the measurement obtained in the average waveform process of block 1513. To this end, a stimulation parameter (e.g., pulse amplitude, pulse width, duty cycle, stimulation frequency) may be decreased in response to the measurement being above a first threshold (high amplitude ECAP response may be outside treatment window). In some embodiments, the pulse width of a stimulation pulse is increased based on an RF signal 130 that is transmitted from the external device, as described above with reference to the methods of FIGS. 7A, 9, 10, and 11. A stimulation parameter may be increased in response to the measurement being below a second threshold (low amplitude or no amplitude ECAP response). In some embodiments, the pulse width of a stimulation pulse is increased based on an RF signal 130 that is transmitted from the external device, as described above with reference to the methods of FIGS. 7A, 9, 10, and 11. The plurality of stimulation parameters may be left at current values in response to the measurement being below a first threshold and above the second threshold.

If none of the stimulation parameters has changed the process returns to block 1501 where the stimulation parameters, e.g., pulse width, pulse amplitude, are reviewed again to confirm that the stimulation level provided by the stimulation parameters is below the activation threshold. The process then cycles through the filtering operation of blocks 1511, 1512, and 1513 for another signal representing sensed electrical activity of a nerve and acquired by the device is processed.

Returning to block 1514, if one or more of the stimulation parameters has changed the process proceeds to block 1515 where the adaptive filter weights are reset or initialized. The filter weights are reset to account for changes to characteristics of the stimulation artifact 1206 that result from changes to the stimulation parameters.

The process then returns to block 1501 where the adjusted set of stimulation parameters are reviewed to determine if the stimulation level provided by the stimulation parameters is below the activation threshold. For example, if stimulation parameters are changed, where the level of stimulus is decreased either by reducing the duty cycle or amplitude for instance, the new set of stimulation parameters may fail to provide enough energy to elicit a nerve response.

At any time during the process of FIG. 15, if the stimulation level is determined at block 1501 to be below the activation threshold, the process enters a learning operation or learning mode. To this end, the process proceeds to block 1502 where filter weights are updated. Algorithms such as a least mean squares (LMS) or recursive least square (RLS) algorithms are examples of routines to update weights of the adaptive filter (applied in block 1511) by minimizing the error between the predicted and measured signal. In a dynamic system where the medium, e.g., neural fibers, may change slightly, for example pressure on the tissue, the characteristics of the medium may change. Therefore, updating the filter weights in real-time when new data is available enhances the performance of the adaptive filter and provides better approximations of the stimulation artifact 1206.

At block 1504, the stimulation level is increased by an incremental amount by adjusting one or more of the stimulation parameters. For example, the level of stimulus may be increased by increasing the duty cycle or amplitude or pulse width for instance. In some embodiments, the pulse width of a stimulation pulse is increased based on an RF signal 130 that is transmitted from the external device, as described above with reference to the methods of FIGS. 7A, 9, 10, and 11. The process returns to block 1501, where if the stimulation level is still below the activation threshold the process cycles through blocks 1502 and 1504 until the activation threshold is reached at block 1501. To ensure physiological information of interest is not filtered, the learning algorithm corresponding to the blocks 1502, 1504 loop is only applied when the stimulation levels are below the activation threshold for an AP response.

To adapt the filter characteristics when switching to filtering mode (blocks 1511, 1512 and 1513) when the stimulation level exceeds the activation threshold at block 1501, a stimulation model is updated which is used to calculate the filter gain for new stimulation settings. This stimulation model can be a simple scalar coefficient that is updated or can utilize a curve fitting function, such as a second order polynomial or linear regression method.

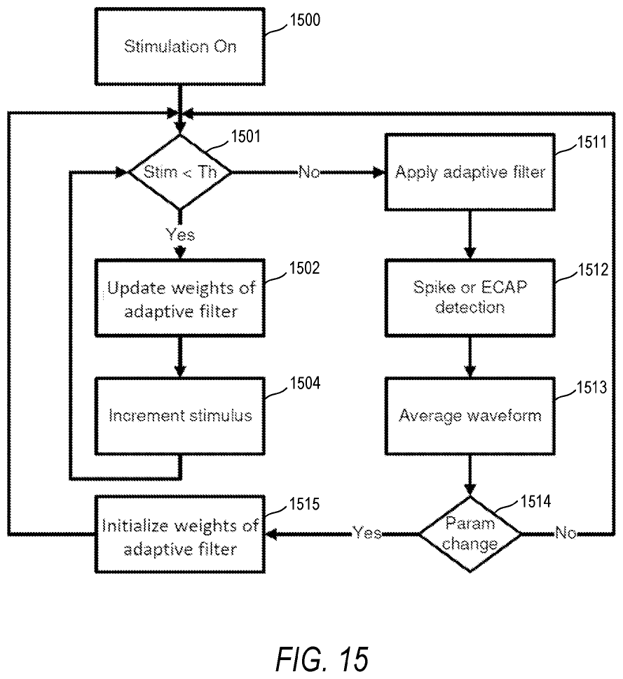

An example of an inverse filter to process a discrete time-series that can be used for fast initialization of a set of Finite Impulse Response (FIR) filter coefficients (used to initialize weights in block 1515) is illustrated in FIG. 16A. The input signal x[k] is convolved with the degradation filter w[k] and the residual is compared to the reference s[k] to calculate an error e[k]. A linear Weiner filter with weights w[k] can approximate the degradation effect from tissue as the stimulation pulse x[k] is applied to bipolar electrodes 1 and 2 discussed earlier. The causal FIR Wiener filter can adopt least squares estimate with input matrix X, output vector y, to determine filter weights derived as follows:

$$W = {( {X}^{T} X )}^{- 1} {X}^{T} y .$$

The output y[k] is the measured signal recorded from bipolar electrodes 3 and 4 described earlier. Such deconvolution filters are typically high-pass and can therefore amplify noise. In such circumstances it is advantageous to add noise to ensure a degree of noise reduction in the filter response by using s[k] to calculate the filter weights. The noise can be Gaussian or bandlimited Gaussian, where the noise signal n[k] is high pass filtered to create noise in the only the higher band, thereby creating attenuation at higher frequencies in the Wiener filter response, having a low pass effect. This requires a block estimation approach that is primarily suitable for initialization purposes.

An example of a real-time adaptive filter (applied in block 1511) is illustrated in FIG. 16B where the filter weights w[k] are adapted based on a learning algorithm such as the Least Means Square (LMS) or Recursive Least Square (RLS) methods. A cost function such as mean square error (MSE) is used to minimize the error between the filter residual y[k] and the desired signal d[k]. The desired signal is the measured stimulation artifact recorded between electrodes 3 and 4 in response to the stimulation waveform x[k]. When the MSE is at a minimum, the coefficients have converged, and the filter adequately models the characteristics of the tissue being stimulated. If the tissue changes due to electrode movement the filter will adapt. Convergence can occur quicker when initialized using the Wiener filter.

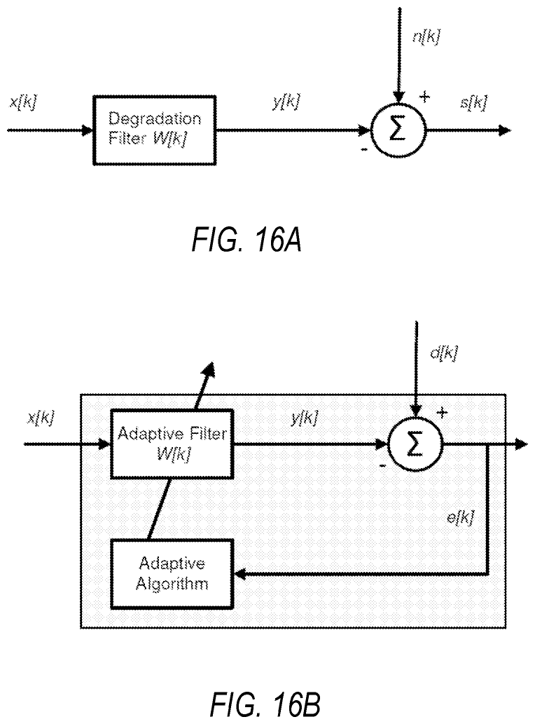

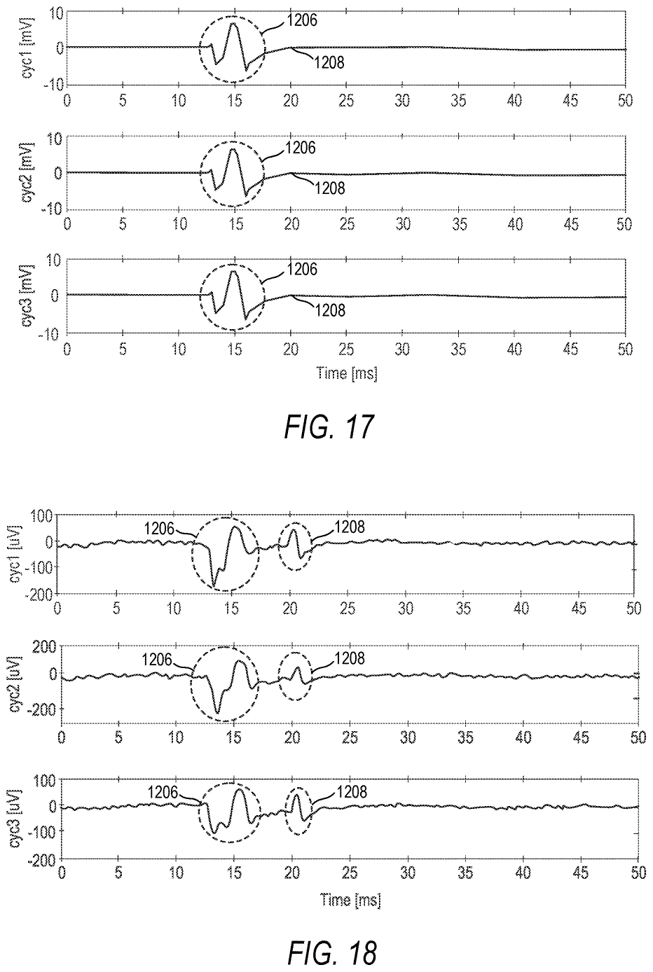

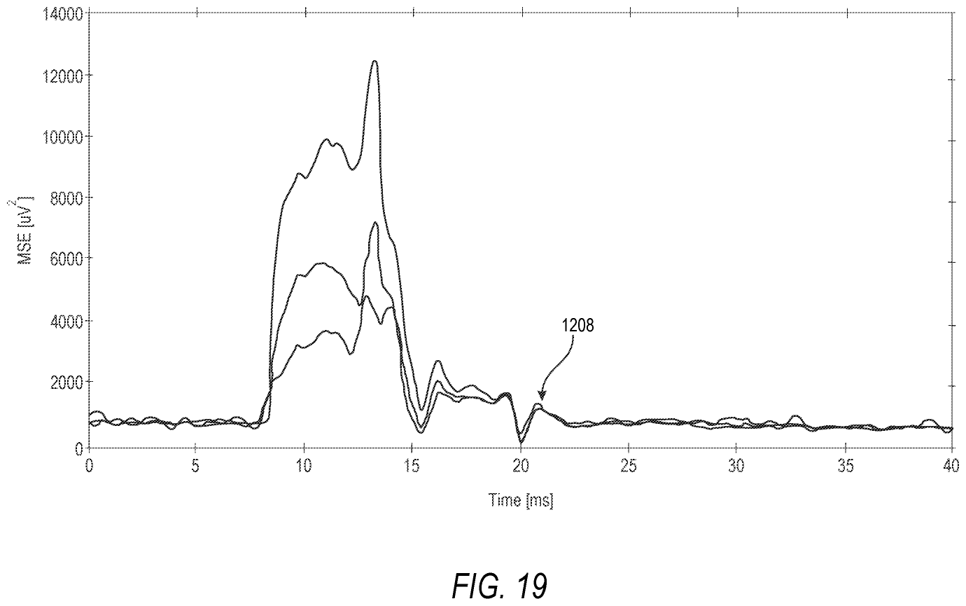

Three stimulation cycles are illustrated in FIG. 17 and FIG. 18 prior and post filtering, respectively. It is evident from FIG. 17 that no ECAPs are visible, and the stimulation artifact 1206 is approximately 7 mV peak-to-peak. With reference to FIG. 18, the result of filtering the stimulation artifact 1206 from the stimulation response 1200 waveforms with the adaptive filter is presented where the stimulation artifact has been reduced to approximately 100 uV peak-to-peak with a reduction of ˜70:1. The AP response 1208 signals are now evident at 20 ms with similar amplitude, and at a level that is identifiable. The AP response 1208 signals can now be processed through spike sorting or template matching algorithms that can be applied to the filter residual to determine the presence and location of the AP response 1208 elicited through stimulation. For example, the results of a template matching routine are illustrated in FIG. 19 for each of the 3 cycles presented, using an MSE measurement to determine fit and location. The MSE approach clearly identifies the AP response 1208 location onset at 20 ms where the error is a minimum for each cycle. This approach could be enhanced by combining the MSE method with a cross-correlation function, although cross-correlation can be erroneously impacted by amplitude.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

1. A neuromodulation system comprising:
a neuromodulation device configured to be implanted at an implant site; and
an external device configured to be positioned at an external sensing site relative to the neuromodulation device,
wherein:
the external device is configured to transmit an RF signal to the neuromodulation device;
the neuromodulation device is configured to deliver a neuromodulation therapy comprising a stimulation pulse at the implant site based on the RF signal; and
the external device is configured to;
obtain a measurement that results from delivery of the stimulation pulse, wherein the measurement is based on an electrical potential sensed between two electrodes in contact with tissue at the external sensing site, and
initiate a change in the neuromodulation therapy based on the measurement, wherein the external device initiates a change in the neuromodulation therapy in response to the measurement being above an upper level by a threshold amount, by changing a configuration of the RF signal to decrease the energy of a subsequent stimulation pulse.

2. (canceled)

3. (canceled)

4. The neuromodulation system of claim 1, wherein the RF signal comprises a notch having a duration, and the external device changes the configuration of the RF signal by reducing the duration.

5. The neuromodulation system of claim 4, wherein the external device reduces the duration of the notch by incrementally reducing the duration until the measurement exceeds the upper level by an amount less than the threshold amount.

6. A neuromodulation system comprising:
a neuromodulation device configured to be implanted at an implant site; and
an external device configured to be positioned at an external sensing site relative to the neuromodulation device,
wherein:
the external device is configured to transmit an RF signal to the neuromodulation device;
the neuromodulation device is configured to deliver a neuromodulation therapy comprising a stimulation pulse at the implant site based on the RF signal; and
the external device is configured to:
obtain a measurement that results from delivery of the stimulation pulse, wherein the measurement is based on an electrical potential sensed between two electrodes in contact with tissue at the external sensing site, and
initiate a change in the neuromodulation therapy based on the measurement, wherein the external device initiates a change in the neuromodulation therapy in response to the measurement being between a lower level and an upper level, by changing a configuration of the RF signal to increase the energy of a subsequent stimulation pulse.

7. The neuromodulation system of claim 6, wherein the RF signal comprises a notch having a duration, and the external device changes the configuration of the RF signal by increasing the duration.

8. The neuromodulation system of claim 7, wherein the external device increases the duration of the notch by incrementally increasing the duration until the measurement exceeds the upper level by an amount less than a threshold amount.

9. A neuromodulation system comprising:
a neuromodulation device configured to be implanted at an implant site; and
an external device configured to be positioned at an external sensing site relative to the neuromodulation device,
wherein:
the external device is configured to transmit an RF signal to the neuromodulation device;
the neuromodulation device is configured to deliver a neuromodulation therapy comprising a stimulation pulse at the implant site based on the RF signal; and
the external device is configured to:
obtain a measurement that results from delivery of the stimulation pulse, wherein the measurement is based on an electrical potential sensed between two electrodes in contact with tissue at the external sensing site, and
initiate a change in the neuromodulation therapy based on the measurement, wherein the external device initiates a change in the neuromodulation therapy in response to the measurement being less than a lower level, by initiating a process to reposition the external device to a different external site.

10. A method comprising:
transmitting an RF signal from an external device at an external sensing site to a neuromodulation device implanted at an implant site;
delivering, by the neuromodulation device, a neuromodulation therapy comprising a stimulation pulse at the implant site based on the RF signal;
obtaining, at the external device, a measurement that results from delivery of the stimulation pulse, wherein the measurement is based on an electrical potential sensed between two electrodes in contact with tissue at the external sensing site; and
initiating, at the external device, a change in the neuromodulation therapy based on the measurement, wherein initiating a change in the neuromodulation therapy comprises, in response to the measurement being above an upper level by a threshold amount, changing a configuration of the RF signal to decrease the energy of a subsequent stimulation pulse.

11. (canceled)

12. (canceled)

13. The method of claim 10, wherein the RF signal comprises a notch having a duration, and changing the configuration of the RF signal comprises reducing the duration.

14. The method of claim 13, wherein reducing the duration of the notch comprises incrementally reducing the duration until the measurement exceeds the upper level by an amount less than the threshold amount.

15. A method comprising:
transmitting an RF signal from an external device at an external sensing site to a neuromodulation device implanted at an implant site;
delivering, by the neuromodulation device, a neuromodulation therapy comprising a stimulation pulse at the implant site based on the RF signal;
obtaining, at the external device, a measurement that results from delivery of the stimulation pulse, wherein the measurement is based on an electrical potential sensed between two electrodes in contact with tissue at the external sensing site; and
initiating, at the external device, a change in the neuromodulation therapy based on the measurement, wherein initiating a change in the neuromodulation therapy comprises, in response to the measurement being between a lower level and an upper level, changing a configuration of the RF signal to increase the energy of a subsequent stimulation pulse.

16. The method of claim 15, wherein the RF signal comprises a notch having a duration, and changing the configuration of the RF signal comprises increasing the duration.

17. The method of claim 16, wherein increasing the duration of the notch comprises incrementally increasing the duration until the measurement exceeds the upper level by an amount less than a threshold amount.

18. A method comprising:
transmitting an RF signal from an external device at an external sensing site to a neuromodulation device implanted at an implant site;
delivering, by the neuromodulation device, a neuromodulation therapy comprising a stimulation pulse at the implant site based on the RF signal;
obtaining, at the external device, a measurement that results from delivery of the stimulation pulse, wherein the measurement is based on an electrical potential sensed between two electrodes in contact with tissue at the external sensing site; and
initiating, at the external device, a change in the neuromodulation therapy based on the measurement, wherein initiating a change in the neuromodulation therapy comprises, in response to the measurement being less than a lower level, initiating a process to reposition the external device to a different external site.

19.-56. (canceled)
